(19) United States
(12) Patent Application Publication
(10) Pub. No.: US 2021/0267770 A1
(43) Pub. Date: Sep. 2, 2021

(54) ACETABULAR IMPLANT ALIGNMENT DEVICES AND METHODS

(71) Applicant: Smith & Nephew Orthopaedics AG, Zug (CH)

(72) Inventors: Marc Patrick Michel Laurent, Münchenstein (CH); Edmond Dauti, Menziken (CH); Martina Kristin Moser, Brugg AG (CH); Florian Tunaj, Schöftland (CH); Marc Guido Oesch, Biberist (CH)

(21) Appl. No.: 17/254,567

(22) PCT Filed: Jun. 26, 2019
(86) PCT No.: PCT/EP2019/066982

§ 371 Date: Dec. 21, 2020

(30) Foreign Application Priority Data
Jun. 26, 2018 (GB) 1810484.4

(51) Int. Cl. A61F 2/46

(52) U.S. Cl. CPC A61F 2/4609; A61F 2002/30143

(57) ABSTRACT

The present invention relates to systems and methods for correctly positioning an acetabular component in an acetabulum of a patient. Particularly although not exclusively embodiments of the present invention relate to an apparatus for aligning an acetabular component in a desired alignment prior to insertion of the acetabular component in the acetabulum. Also disclosed herein are systems which comprises an apparatus (1000, 1100) for inserting an acetabular component and an apparatus (100) for locating the acetabular component during the insertion operation.

### FIELD OF THE INVENTION

The present invention relates to systems and methods for correctly positioning an acetabular component in an acetabulum of a patient. Particularly although not exclusively embodiments of the present invention relate to an apparatus for aligning an acetabular component in a desired alignment prior to insertion of the acetabular component in the acetabulum. Also disclosed herein are systems which comprise an apparatus for inserting an acetabular component and a device for locating the acetabular component during the insertion operation. Aptly, the systems and apparatus of embodiments of the present invention may be suitable for use in combination with cemented and non-cemented acetabular components. Aptly, the system and apparatus described herein are for use in total hip replacement surgery.

### BACKGROUND TO THE INVENTION

Hip replacement is a surgical procedure in which the hip joint is replaced by a prosthetic implant. Total replacement of the hip joint involves installing an acetabular cup implant in the acetabulum of a patient and installing a prosthetic in the femur of the patient. A ball of the prosthetic is received in the acetabular cup implant.

Successful hip replacement surgery requires correct alignment and positioning of the acetabular cup implant. Misalignment of the acetabular component may result in dislocation, undesired wear and tear of the bearing surfaces of the implants and/or restricted movement of the prosthetic within the acetabulum.

Hip replacement surgery sometimes requires the fixation of the acetabular cup implant with cement in the acetabulum. Cement can escape from the acetabulum when the implant is being positioned.

There is a need to provide means to correctly align acetabular components within an acetabulum during surgery and also to avoid displacement of cement in cemented procedures.

### SUMMARY OF THE INVENTION

Various aspects and embodiments of the present invention are described herein.

In a first aspect of the present invention, there is provided an apparatus for locating an acetabular component in an acetabulum of a patient, the apparatus comprising:

a. a proximal portion;

b. a distal portion; and

c. a connecting portion intermediate the proximal portion and the distal portion, wherein the connecting portion includes:
i) an arm portion connected to the distal portion and comprising a longitudinal axis; and
ii) an alignment guide portion extending between the proximal portion and the arm portion, the alignment guide portion being integrally formed with the proximal portion and the distal portion and comprising a first branch portion and a second branch portion, and wherein the first branch portion and/or the second branch portion provides an alignment surface.

In certain embodiments, the acetabular component comprises a polar axis and the apparatus serves to orient the polar axis at a prescribed angle of inclination and a prescribed angle of anteversion.

In certain embodiments, when the patient is located in a supine or lateral position on an operating table, the patient having a long axis, the alignment surface is configured to be locatable at a predetermined angle with respect to the long axis of the patient so as to orient the polar axis at the prescribed angle of inclination.

In certain embodiments, the alignment surface comprises a plane and wherein the plane of the alignment surface is generally parallel to the coronal plane of the patient to represent an angle of anteversion of between 10° to 20° e.g. about 15°. In certain embodiments, the first branch portion comprises a first alignment surface configured to align the acetabular component in a first acetabulum of the patient and the second branch portion comprises a further alignment surface configured to align the acetabular component in a further acetabulum of the patient.

In certain embodiments, the apparatus serves to orient the acetabular component along an alignment axis in the acetabulum during surgery, wherein the alignment axis is an axis along which it is desirable to insert an acetabular component into the acetabulum during surgery.

In certain embodiments, the alignment axis extends at an angle of:
about 40 to 50° inclination from the centre of the acetabulum; and
an angle of 0 to 30° anteversion from the centre of the acetabulum; e.g. an angle of about 5 to about 25° e.g. about 10 to 20° anteversion from the centre of the acetabulum.

In certain embodiments, the alignment guide portion comprises a central region from which the first branch portion and the second branch portion extend at an acute angle to each other, the acute angle being in the range of from about 15° to about 60°. In certain embodiments, the acute angle is about 35° to about 45°. In certain embodiments, the acute angle is about 40°.

In certain embodiments, the first branch portion and the second branch portion are substantially symmetrical about a longitudinal axis of the alignment guide portion. In certain embodiments, the arm portion comprises a longitudinal axis and further wherein the longitudinal axis of the alignment guide portion and the longitudinal axis of the arm portion are generally orthogonal with respect to each other.

In certain embodiments, the proximal portion comprises a handle portion at a proximal end thereof. In certain embodiments, the distal portion comprises an insertion portion at a distal end thereof, wherein the insertion portion comprises a plate portion having a central protrusion.

In certain embodiments, the plate portion is generally circular. In certain embodiments, the central protrusion comprises a conical frustum. In certain embodiments, the apparatus is an integrally formed apparatus.

In certain embodiments, the apparatus further comprises a detachable introducer plate configured to be locatable in an acetabular component.

In certain embodiments, the apparatus is configured to locate a cemented acetabular component in the acetabulum of a patient.

In a further aspect of the present invention, there is provided a method of locating an acetabular component in an acetabulum of a patient, the method comprising:
a). providing an apparatus according to the first aspect of the present invention;
b) locating an acetabular component at the distal end of the apparatus; and
c) advancing the apparatus to locate the acetabular component in the acetabulum of the patient.

In certain embodiments, the method further comprises locating a fixation material in the acetabulum of the patient prior to step (c).

In certain embodiments, the method comprises aligning the first branch region or the second branch region such that the plane of the respective alignment surface is generally parallel to the coronal plane of the patient, when the patient is positioned in an extended supine or lateral position. In certain embodiments, the method further comprises aligning the arm portion generally orthogonally to the longitudinal axis of the patient, when the patient is positioned in an extended supine or lateral position.

In certain embodiments, the method further comprises withdrawing the apparatus from the acetabulum once the acetabular component is located in a desired orientation in the acetabulum.

In certain embodiments, the method further comprises, prior to step (c), the steps of:
a) incising a hip with an incision; and
b) exposing at least a portion of an acetabular joint through the incision.

In a further aspect of the present invention there is provided a kit comprising the apparatus according to the first aspect of the present invention, and at least one acetabular component.

In certain embodiments, the acetabular component is a trial component or an acetabular prothesis. In certain embodiments, the kit further comprises a fixation material e.g. cement.

In a further aspect of the present invention there is provided a device for introducing an acetabular component into an acetabulum of a patient; the device comprising:
a) a base portion comprising a central aperture;
b) a radially expandable upper portion having a lower surface which is at least partially connected to the base portion, the upper portion comprising at least one slit and a central cut-out region, wherein the cut-out region of the upper portion and the central aperture of the base portion are at least partially aligned.

In certain embodiments, the at least one slit is configured to enable the upper portion to expand or contract in circumference upon application of pressure.

In certain embodiments, the upper portion comprises at least two slits. In certain embodiments, the upper portion is a truncated cylinder. In certain embodiments, the base portion comprises an outer annular rim portion.

In certain embodiments the device is an introducer plate.

In a further aspect of the present invention, there is provided a system comprising the apparatus according to the first aspect of the present invention and a device according to a further aspect of the present invention, wherein the central protrusion is configured to be locatable in the central aperture of the base portion and the cut-out region of the upper portion.

In a further aspect of the present invention there is provided a system for locating an acetabular component in an acetabulum of a patient, the system comprising:

a) an apparatus for introducing the acetabular component into the acetabulum, the apparatus comprising:
i) a proximal portion comprising a handle section adapted to support a first strike plate,
ii) a distal portion; and
iii) a connecting portion intermediate the proximal portion and the distal portion; and

b) a device for supporting an acetabular component during the locating operation, the device comprising a base portion having a central aperture and a radially expandable upper portion having a lower surface which is at least partially connected to the base portion, the upper portion comprising at least one slit and a central cut-out region, wherein the cut-out region of the upper portion and the central aperture of the base portion are at least partially aligned.

In certain embodiments, the device is a device as described herein.

In certain embodiments, the distal portion of the apparatus comprises a plate for mounting the device. Particularly, the plate may comprise a protrusion locatable in the central cut-out position and the central aperture to secure the device to the apparatus. In some embodiments, the system further comprises an acetabular component locatable over the upper portion of the device such that a rim portion of the acetabular component abuts the base portion. The apparatus may be an apparatus according to a first aspect of the present invention or may be an introducer apparatus as described herein. In certain embodiments, the connecting portion comprises a lever element which is connected to the central protrusion.

Also included in the present invention is an apparatus for locating an acetabular component in an acetabulum of a patient, the apparatus comprising a proximal portion; a distal portion; and a connecting portion intermediate the proximal portion and the distal portion, wherein the connecting portion includes an arm portion connected to the distal portion and comprising a longitudinal axis; and an alignment guide portion extending between the distal portion and the arm portion, the alignment guide portion being integrally formed with the proximal portion and the distal portion and comprising a first branch portion and a second branch portion, and wherein the first branch portion and/or the second branch portion provides an alignment surface.

In a yet further aspect of the present invention there is provided an apparatus for introducing an acetabular component into an acetabulum of a patient, the apparatus comprising:
i) a proximal portion comprising a gripping portion;
ii) a distal portion comprising a securing element comprising a frusto-conical surface; and
iii) an elongate body portion extending between the proximal portion and the distal portion, the elongate body portion comprising a lever element pivotally secured to the elongate body portion, wherein the lever element is pivotable to drive the frusto-conical surface of the securing element between a first position away from the elongate body portion for receiving an acetabular component and/or an introducing plate as described herein and a further position in which the frusto-conical surface is located proximate to an end of the elongate body portion to secure an acetabular component and/or an introducing plate as described herein at an end of the elongate body portion.

In certain embodiments, the elongate body portion further comprises a drive arm pivotably connected to the lever element and to the securing element. In certain embodiments, the drive arm is housed within the elongate body portion. In certain embodiments, the elongate body portion comprises a cylindrical extension at a distal end thereof. In certain embodiments, a circular base plate is located at the distal end of the elongate body portion which is mounted onto the cylindrical extension.

In certain embodiments, the securing element further comprises a projection which extends towards the elongate body portion. In certain embodiments, the projection is hollow and comprises a bore sized to accommodate a portion of the drive arm. In certain embodiments, the portion of the drive arm is located within a portion of the elongate body portion.

In certain embodiments, the elongate body portion further comprises at least one cut-out portion.

In certain embodiments, the apparatus further comprises a handle element connectable to the elongate body portion. In certain embodiments, the handle portion comprises a hexagonal-shaped end portion. In an embodiment, the handle portion comprises a protrusion which is configured for location in an aperture in an upper surface of the elongate body portion.

In certain embodiments, the protrusion is spring-loaded.

In certain embodiments, the elongate body portion comprises a further aperture on an upper surface thereof.

In certain embodiments, the elongate body portion is arcuate. In certain embodiments, the elongate body portion is substantially straight.

In certain embodiments, the lever element comprises an arcuate portion and a flattened portion, wherein the flattened portion is configured to locate at least partially within a recessed portion of the grippable portion.

In certain embodiments, the lever element comprises a straight portion and a flattened portion wherein the flattened portion is configured to locate at least partially within a recessed portion of the grippable portion.

In a further aspect of the present invention there is provided a system comprising the device as described herein and an Introducer apparatus as described herein.

### BRIEF DESCRIPTION OF DRAWINGS

Embodiments of the present invention will now be described hereinafter, by way of example only, with reference to the accompanying drawings in which:

FIG. 1 helps to illustrate the components of a system according to certain embodiments of the present invention.

FIGS. 2A to 2D illustrate an apparatus according to certain embodiments of the present invention

FIG. 3 helps to illustrate various components of a system and kit of certain embodiments of the present invention;

FIG. 4 illustrates a combination of a device and apparatus according to certain embodiments of the present invention;

FIG. 5 illustrates an alternative view of a combination of a device and apparatus according to certain embodiments of the present invention;

FIG. 6 illustrates a method of assembling a system according to certain embodiments of the present invention;

FIG. 7 illustrates various components of a system of certain embodiments of the present invention;

FIG. 8 illustrates steps of methods according to certain embodiments of the present invention;

FIGS. 9A and 9B are cross-sectional views of a device and apparatus according to certain embodiments of the present invention;

FIGS. 10A, 10B and 10C show a device (introducer plate) according to certain embodiments of the present invention;

FIGS. 11A to 11D illustrate an introducer apparatus of systems according to certain embodiments of the present invention;

FIGS. 11E and 11F illustrate a further introducer apparatus according to certain embodiments of the present invention which is for use in a cemented procedure;

FIGS. 11G and H illustrate a further introducer apparatus according to certain embodiments of the present invention which is for use in a cementless procedure;

FIG. 12 is a cross sectional view of an introducer plate according to certain embodiments of the present invention mounted on an end portion of the introducer apparatus of FIG. 11;

FIG. 13 is a cross sectional view of a lever of the introducer apparatus of FIG. 11;

FIG. 14A illustrates an introducer apparatus of systems according to certain embodiments of the present invention, wherein the apparatus comprises a non-self locking lever;

FIG. 14B illustrates an introducer apparatus of systems according to certain embodiments of the present invention with a device according to certain embodiments mounted thereon;

FIG. 15 illustrates an introducer apparatus with a device according to certain embodiments mounted thereon;

FIG. 16 illustrates the introducer apparatus of FIGS. 11G and 11H.

### REFERENCE NUMERALS

1—device (introducer plate)
100—apparatus (alignment apparatus)
200—acetabular shell (for cement)
300—acetabular trial shell
400—acetabular shell (cementless)
500—acetabular shell (cementless)
1000—introducer apparatus
1100—introducer apparatus
1200—introducer apparatus
1300—introducer apparatus

### DETAILED DESCRIPTION

As used herein, the term “distal” refers to components which are distal with respect to the surgeon or user of the apparatus. The term “proximal” refers to components which are proximal with respect to the surgeon or user of the apparatus. Unless indicated otherwise, like numerals relates to like parts.

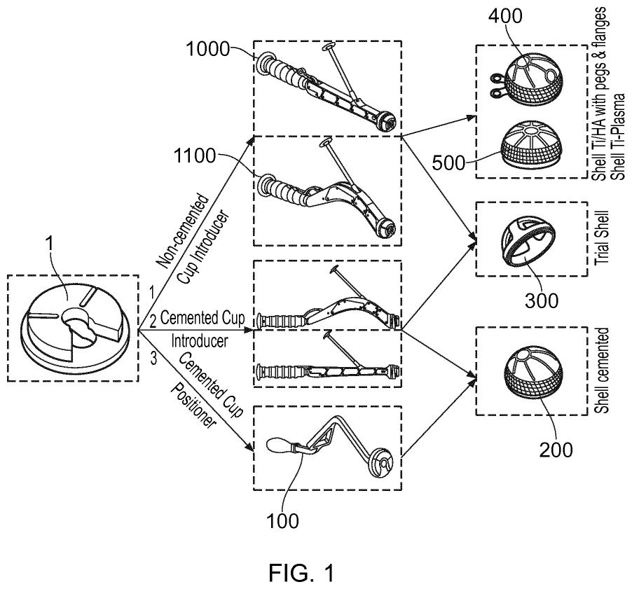

FIG. 1 illustrates the various components of a system according to certain embodiments of the present invention. Particularly, the system may comprise a device 1. The device may also be referred to herein as an introducer plate. The introducer plate can be used in combination with an apparatus 100 as described herein. Alternatively or in addition, the system may comprises one or more introducer apparatus 1000, 1100, 1200, 1300.

Furthermore, the system may comprise an acetabular component 200, 300, 400, 500. The acetabular component may be a component known in the art such as for example a trial prosthesis or an acetabular prosthesis or the like.

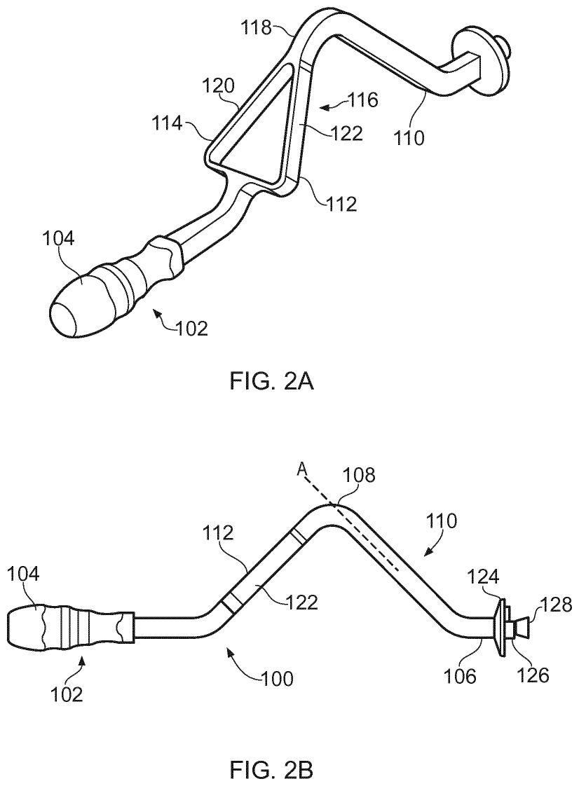

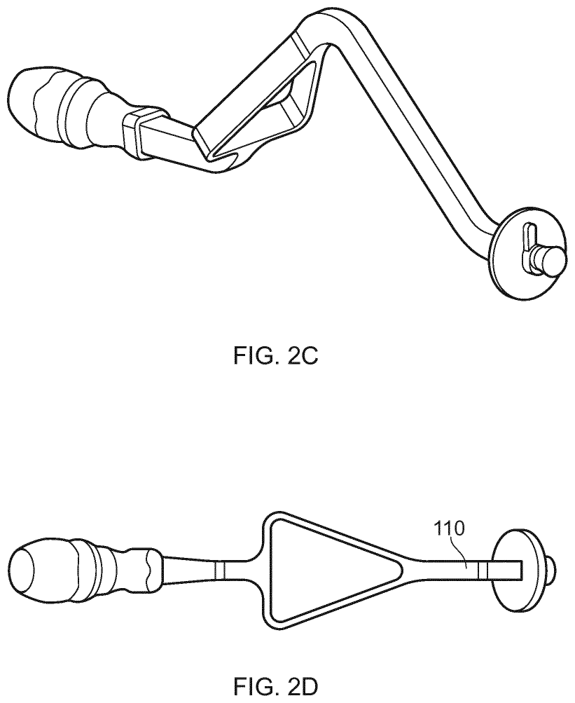

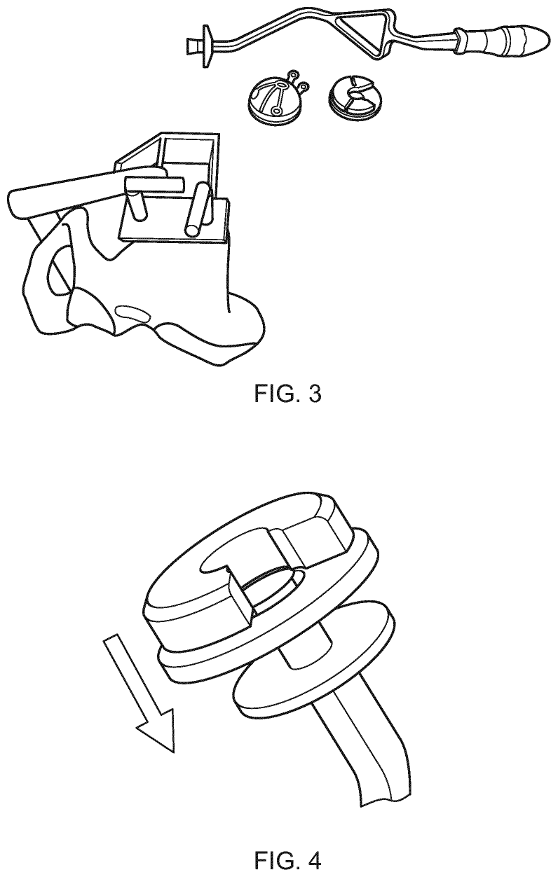

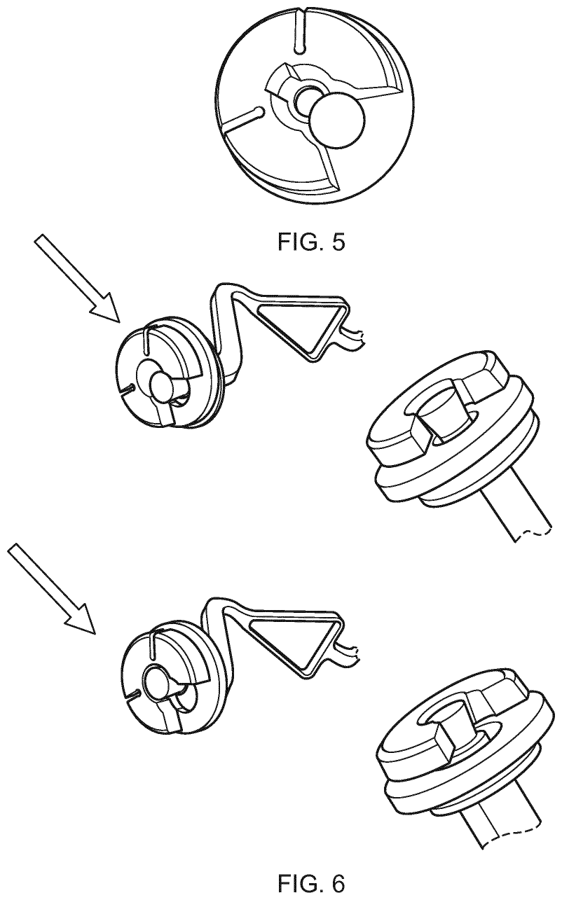

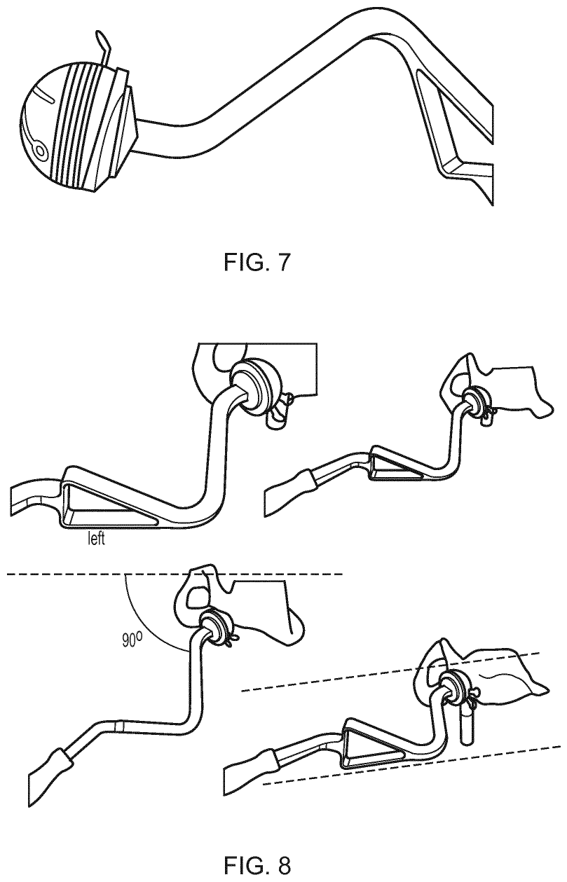

As shown in FIG. 2, certain embodiments relate to an apparatus 100 for aligning an acetabular component in a patient's acetabulum. The illustrated apparatus is configured for use in combination with a device according to certain embodiments described herein. It will be understood that in certain embodiments the apparatus is for use with an alternative introducer plate.

The apparatus 100 comprises a proximal portion 102 which comprises a grippable handle portion 104. The apparatus further comprises a distal portion 106 which is advanced towards the surgical site in the patient's acetabulum during use. The distal portion and the proximal portion may comprise a longitudinal axis in a common plane.

The apparatus further comprises a connecting portion 108 intermediate the proximal portion and the distal portion. The connecting portion is elongate and may be hollow in certain embodiments. The connecting portion comprises an arm portion 110 which is connected to the distal portion 106. The arm portion comprises a longitudinal axis (A). The connecting portion 108 further comprises an alignment guide portion 112 extending between the proximal portion and the arm portion. Aptly, the alignment guide portion 112 is integrally formed with the proximal portion 102 and the distal portion 106. The alignment guide portion 112 comprises a first branch portion 114 and a second branch portion 116. The alignment guide portion therefore comprises a triangular shaped section as shown in FIG. 2A.

The alignment guide portion also includes a central portion 118 from which the first branch portion and the second branch portion extend generally symmetrically e.g. at an angle of around 40° from each other. The first branch portion 114 comprises at least one alignment surface 120 which can be used to align the positioning apparatus 100 with the coronal plane of the patient on the operating table. The patient is in the supine or lateral position on the operating table. Particularly and as described below, the plane of the alignment surface 120 of the first branch portion is aligned parallel to the patient's coronal plane in order to align an acetabular component at between 10 to 20° anteversion in the patient's prepared acetabulum. The alignment surface 120 of the first branch portion is used to align an acetabular component in the right hip. The first branch portion 114 may include a visual indicator e.g. “RIGHT” to show the surgeon to use the alignment surface 120 for aligning an acetabular component in the right hip.

The second branch portion also comprises an alignment surface 122. The alignment surface 122 can be used to align an acetabular component in an acetabulum of the patient with between around 10 to 20° anteversion. The plane of the alignment surface 122 of the second branch portion is aligned parallel to the patient's coronal plane in order to align an acetabular component at around e.g. 20° anteversion in the patient's prepared acetabulum. The alignment surface 122 of the second branch portion is used to align an acetabular component in the left hip. The second branch portion 116 may include a visual indicator e.g. “LEFT” to show the surgeon to use the alignment surface 122 for aligning an acetabular component in the left hip.

The first and second branch portions are integrally formed with both the proximal portion i.e. a handle portion, and the distal portion i.e. the insertion end region. As a result, the apparatus of certain embodiments may be easy to use and manufacture. The apparatus as described herein does not contain sharp ends, which may help to reduce injury during surgery. Furthermore, the apparatus of certain embodiments may be sterilised as a single piece and re-used.

The arm portion is generally orthogonal to the alignment guide portion. The arm portion is generally linear and elongate. The arm portion can be used during surgery to align the acetabular component in a correct inclination in the acetabulum. Particularly, the longitudinal axis (A) of the arm portion is aligned at 90° relative to the axis of the patient, when the patient is in the supine or lateral position on an operating table. The alignment of the arm portion orthogonal (i.e. 90°) to the patient's longitudinal axis typically results in the acetabular component being inclined at 45° in the patient's acetabulum.

As shown in FIGS. 2A and 2B, the longitudinal axis of the arm portion is generally at an angle of about 90° to the longitudinal axis of the alignment guide portion.

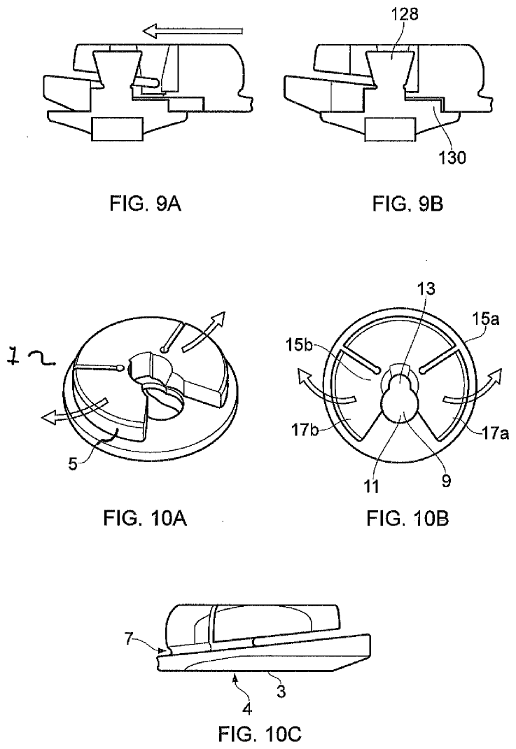

Also shown in FIGS. 2A and 2B for example, the distal portion 108 comprises an insertion flange portion 124. The insertion flange portion is circular and comprises a central protrusion 126 which has a cone-shaped end portion 128 and a stepped portion 130. The cross-section of the insertion flange portion is shown in FIGS. 9A and 9B.

In some embodiments, the protrusion 126 is configured to fit within an aperture of an insertor plate as described herein. In certain embodiments, the apparatus is integrally formed as a unitary body.

### Introducer Plate

Certain embodiments of the present invention provide a device for introducing an acetabular component into an acetabulum of a patient. FIG. 10 illustrates a device 1 of certain embodiments. The device 1 (also referred to herein as an “introducer plate”) comprises a lower portion 3 having a flat lower surface 4 which abuts an upper surface of the introducer flange of the apparatus in use. The introducer plate is generally circular and comprises an upper protrusion 5 which is spaced apart from the lower portion at least partially and is attached to the lower portion via a stem portion 7. The upper protrusion has a generally flat upper surface.

The introducer plate comprises a central aperture 9 which is substantially keyhole shaped. A lower portion 11 of the central aperture has a wider diameter than the upper portion 13.

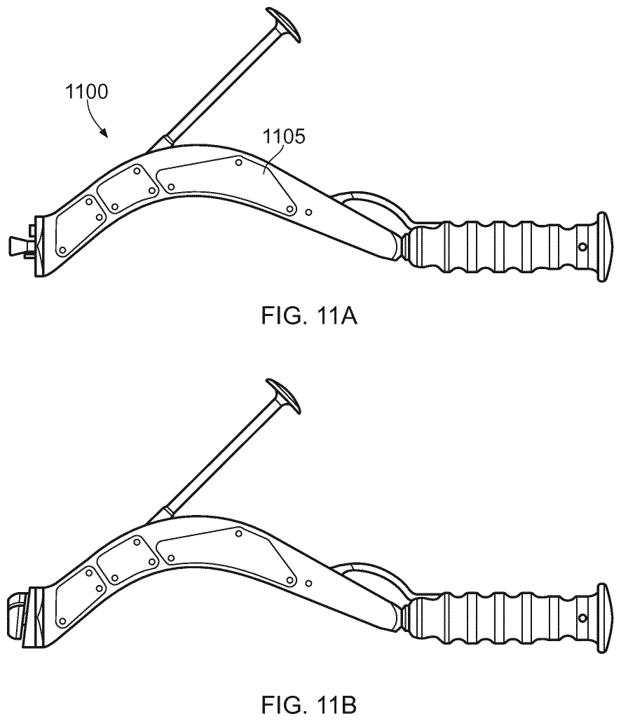

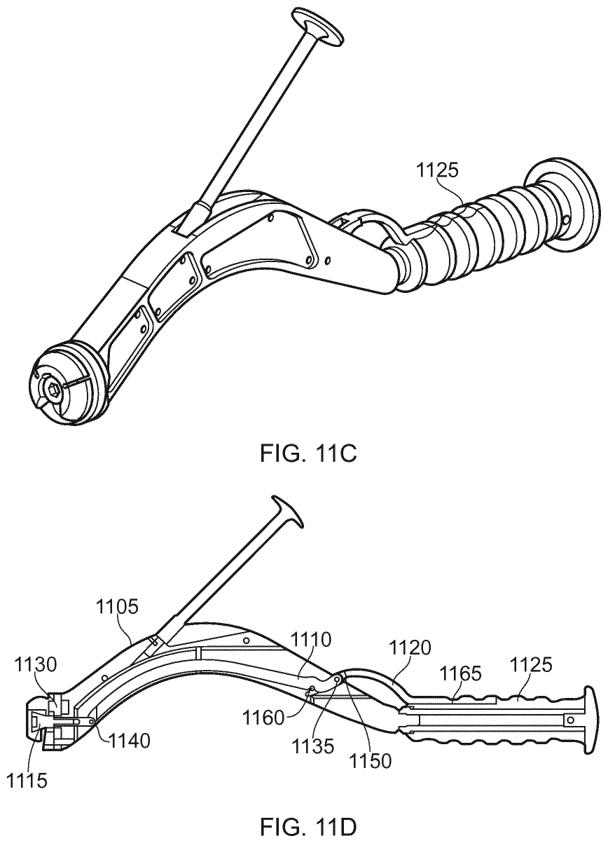

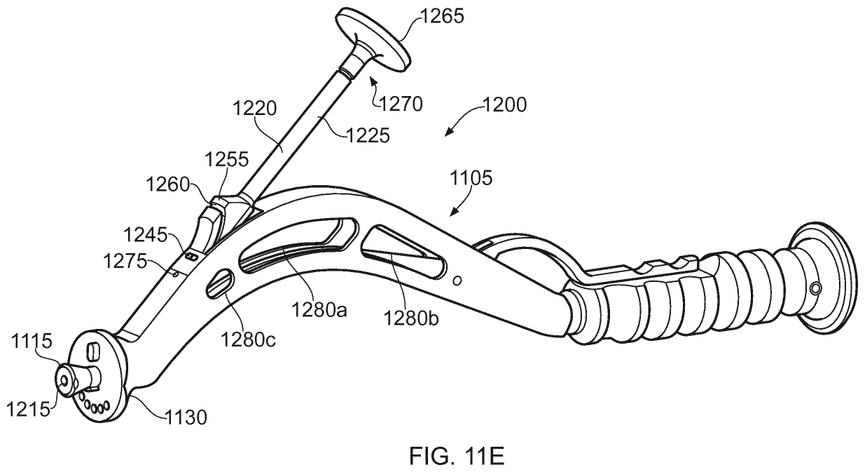

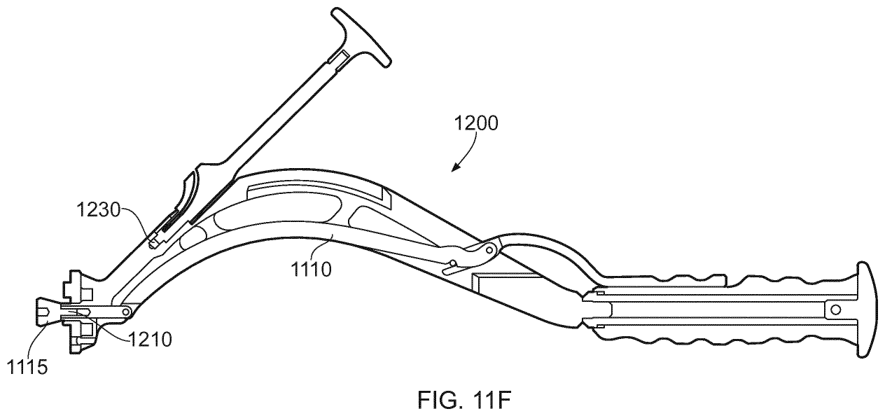

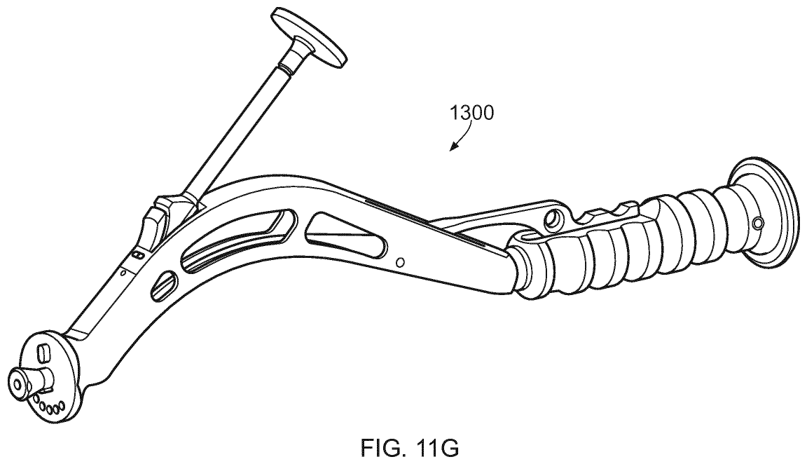

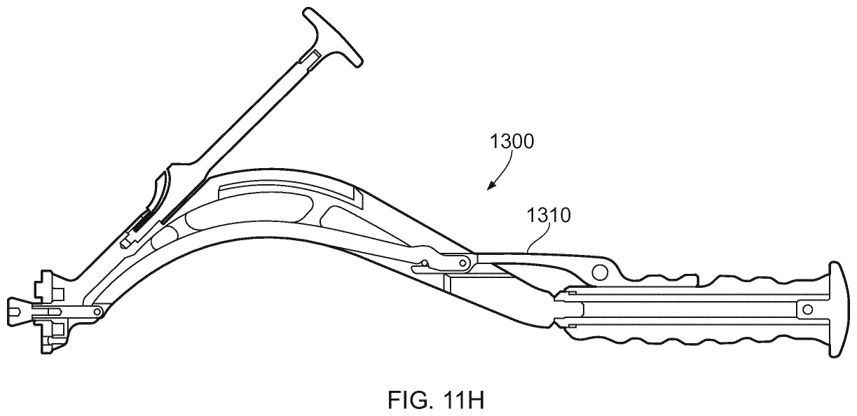

The upper protrusion 5 is generally circular and comprises at least one slit. The illustrated embodiment comprises two slits 15a, 15b. The slits extend from the outer perimeter of the upper protrusion towards but not extended into the central aperture. The slits form at least two wing portions e.g. 17a and 17b as shown in FIG. 10B. The upper protrusion 5 is radially expandable as shown by the arrows in FIG. 10A. The inserter plate may be mounted to the apparatus as shown in FIG. 11.

The inserter plate may be formed from any suitable material including for example plastic. The use of the inserter plate may confer a number of advantages over existing technologies including for example it enables fixation of a non-cemented acetabular component (“cup”) with a strong retention force and good load transmission on the inside of the cup and on the ring when impacting the cup. Additionally, it may enable fixation of a cemented cup with a strong retention force when introducing and aligning the cup and also be completely loose from the cup when releasing the instrument. In addition, the cup may be fully covered to prevent cement inflow. For embodiments in which the introducer plate and system is used to implant a cemented acetabular component, the retention force of the cup on the Introducer Plate is fully controlled by the surgeon's hand. There is little or no movement of the cup when the apparatus is withdrawn from the surgical site. Furthermore, it may provide a loose connection for a cemented cup but fully covers the cup to prevent cement inflow.

In certain embodiments, the introducer plate may comprise one or more visual cues e.g. a notch (not shown) which can be aligned with a corresponding indicator on an acetabular component. In certain embodiments, the geometry of the introducer plate exactly fits into the inner geometry of the cup including the 6° cylindrical cover. The introducer plate covers the rim of an acetabular component. In certain embodiments, the inserter plate can be used with an introducer apparatus described below. In these embodiments, a lever provided by the introducer apparatus can tighten the fit of the introducer plate within the acetabular component.

The central protrusion of the apparatus is designed to allow coupling with the Introducer Plate. The central protrusion of the apparatus is fixed and static unlike the central protrusion of an introducer apparatus described below. When the Introducer Plate is mounted onto the apparatus, the cup has a loose fixation. The outer annular rim of the introducer plate is configured to contact the rim of the acetabular component and thus allows aligning the acetabular prosthesis according to desired angles of inclination and anteversion to the indicated angles.

The central protrusion is passed through the keyhole shaped aperture at its greatest diameter. The Introducer plate is then slid downwardly so that the central protrusion is accommodated in and through the keyhole shaped aperture at its smaller diameter, thus enabling the central protrusion to fit in the middle of the Introducer Plate.

### Introducer Apparatus

In some embodiments, the introducer plate may be for use with an apparatus for inserting an acetabular component other than the apparatus of the first aspect. For example, the introducer plate may be for use in a system which comprises an apparatus for inserting an acetabular component such as the introducer apparatus 1000 and 1100 shown in FIG. 1. The introducer apparatus can be those known in the art.

The Introducer apparatus can be used in at least four different configurations. For example in certain embodiments, the Introducer apparatus 1100 may be curved as shown in FIGS. 11A and 11B. A further embodiment is shown in FIGS. 11E and F for example. The curved Introducer apparatus may be configured for cementless or cemented acetabular prosthesis implantation.

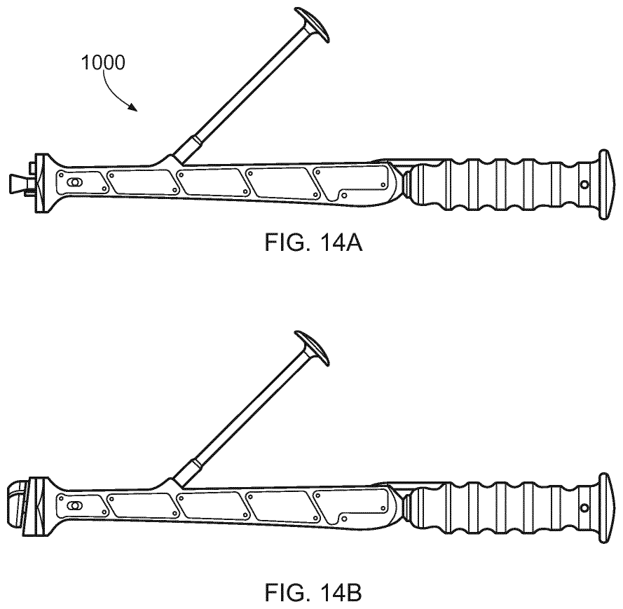

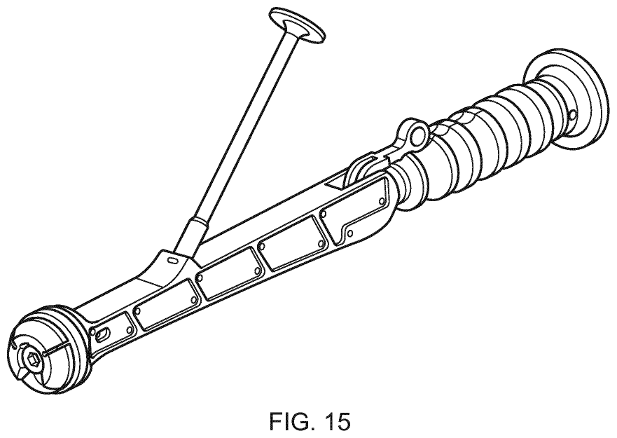

An alternative embodiment of an Introducer apparatus 1000 is shown in FIGS. 14A, 14B, 15A and 15B. The Introducer apparatus comprises a straight shaft and may be configured for cemented or cementless acetabular prosthesis implantation.

As shown in FIGS. 11A and 11B, 11E, 11F and 15A and 15B for example, each configuration is made of a base body 1105, whether a curved or straight one, and an inner-lever 1110. Aptly the inner lever can differ depending on whether it is for use with cementless or cemented implantation of an acetabular component. Thus, the introducer apparatus 1100, 1000, 1200, 1300 comprises a base body which is empty and ready to be coupled with an inner-lever 1110. The inner-levers are easily dismountable from the base bodies for modularity and for cleanability purposes. The inner lever may also be referred to as a drive arm herein.

The inner lever extends from an entry port 1150, shown in FIG. 11D through the base body and attaches to a central protrusion 1115 at a distal end of the apparatus. In certain embodiments, the inner-lever has one extremity attached to a securing element. An example of a securing element is shown in FIG. 11D as a protrusion 1115 e.g. a cone and another extremity 1120 which is manipulated by the user during surgery. The cone is provided at a distal end of the apparatus, where the Introducer Plate is mounted on the Introducer apparatus. The securing element may also comprise a projection 1210 connected to or integrally formed with the cone. The projection 1210 is generally cylindrical and sized to fit within an open bore provided by a cylindrical extension of the base body. The bore of the cylindrical extension also accommodates a cylindrical distal portion of the drive arm. The open bore may include a shoulder portion to enable a user to guide the cylindrical distal portion of the drive arm through the bore.

The projection 1210 and the outer surface of the cylindrical distal portion of the drive arm are threaded to enable the cone to be secured to the drive arm.

By pulling the inner-lever in a direction towards a first handle 1125 of the apparatus to the handle, the user pulls the cone toward the Introducer plate causing its “wings” to spread and allows a tight coupling with the acetabular cup placed on it. The first handle 1125 comprises a grippable surface.

The cone of the embodiment shown in FIG. 11E comprises a hexagonal-shaped recess 1215 which is sized for use with an “Allen” wrench element provided on the handle 1220 described below. The Allen wrench may be placed in the recess and used to unscrew the cone if the surgeon considers the cone to be too tight following actuation of the inner lever. This provides an advantage of being able to unscrew the cone during and/or after the surgical procedure. The Allen wrench element may also make it easier to remove the cone for cleaning purposes after use.

As mentioned above, the Introducer base body can be coupled with an inner-lever for cementless implantation of the acetabular components. In this case the inner-lever is designed to be a self-locking lever, which means that when tightened, it doesn't need to be held and will stay locked even when the user removes their hand from handle. An example of an apparatus comprising a self-locking lever is shown in FIG. 15. An example of an apparatus comprising a non-self locking lever is shown in FIG. 11. Aptly, the non self-locking lever is for use to implant cemented acetabular components.

The apparatus comprises a plate 1130 to which the cone is fixed. The Inserter plate rests against the plate 1130 when positioned over the cone. Furthermore, the apparatus comprises a fixation point 1140 where the inner-lever is pivotably attached to the Introducer base-body.

In the context of a non-self-locking inner lever as shown in FIG. 11D, when the user manipulates the external portion of the lever 1120, the fixation point 1135 stays above an imaginary line which intersects the two fixation points 1140 and 1135.

The inner-lever or drive arm is provided inside the base body via an opening shown in FIG. 11D. The left extremity of the inner-lever passes through a hole at the left end of the base body where the cone is then screwed to it. This action fixes one extremity of the inner-lever to the base body. A second extremity of the inner-lever is fixed to the base body as shown in FIG. 13. A pin 1160 is provided (as shown in FIGS. 11D and 13) which extends through the base body and is attached to it. The inner-lever is clipped to this pin and stays attached to it due to its shape which acts like a spring.

The self-locking lever is shown in FIG. 15 for example and is locked to the handle until a user releases it. The self-locking lever is pivotably connected to the base body via a fixation point (not shown). The self locking lever comprises a portion which is external to the base body, similar to external portion 1120 of the non-self locking lever. When the user moves the external portion of the lever downwardly towards the handle, the fixation point moves below an imaginary line which intersects two fixation points which correspond to 1140 and 1135.

The self locking lever further comprises an eyelet on the external portion thereof which can be used to release the lever from the handle when required.

In certain embodiments, the introducer plate can be used with an Introducer apparatus as described herein for cemented implantation of an acetabular component. For example, the introducer plate can be coupled with an apparatus which comprises an inner lever which is not a self-locking lever. In this embodiment, the inner-lever needs to be held by a user at an end portion thereof adjacent to the handle portion. The user grips both the end portion 1165 and the handle simultaneously. The end portion and therefore the inner lever will be released (open) when the user removes their hand from the handle. This embodiment is particularly useful when implanting with cement, as the acetabular component must be held still.

Conversely when using a self-locking inner-lever, the release of the shell is only possible by unlocking the self-locking mechanism, an action which causes little shocks which are not desirable for implantation with cement. Thus, a non-self-locking mechanism allows a very smooth and controlled release of the shell without little shocks.

Aptly, when an introducer apparatus is used in the system, the central protrusion or equivalent cone is mobile and when tightened, the cone presses against the Plate which has the effect to spread the wing portions of the introducer plate toward the outside. The wing portions are considered to be the end portions of the upper protrusion. When spreading toward the outside they are moving in direction of the arrows shown in FIG. 10B.

When an acetabular component is placed on the introducer plate, the spreading of the wing portions creates a pressure on the inner rim of the shell allowing a tight coupling between the Shells and the Plate and thus between the Shells and the Introducer apparatus. When releasing the force on the wing portions, the connection between Cup and Introducer Plate is completely loose. Depending on the used mechanism of moving the cone, either self-locking or full control of applied force by the user can be achieved.

In certain embodiments, the more force is applied on the actuating handle (e.g. a lever), the more the Introducer Plates wings are spread, the more the cup is engaged. If the force on the lever is removed, the cup is completely loose.

In certain embodiments, the introducer apparatus comprises a second handle 1220 that is detachable from the base body 1105. The handle is attachable to the base body generally towards the distal region thereof. The handle comprises a shaft 1225 having a longitudinal axis. The longitudinal axis of the handle shaft provided at an angle of between about 35° and about 70° to a longitudinal axis of the Introducer apparatus. In some embodiments the angle is between about 45° and about 60°.

The handle may comprise a distal end region which includes an end portion 1230 that includes a hexagonal or “Allen”-type wrench portion. The Allen wrench portion is designed to interact with the recess of the cone 1215 described above.

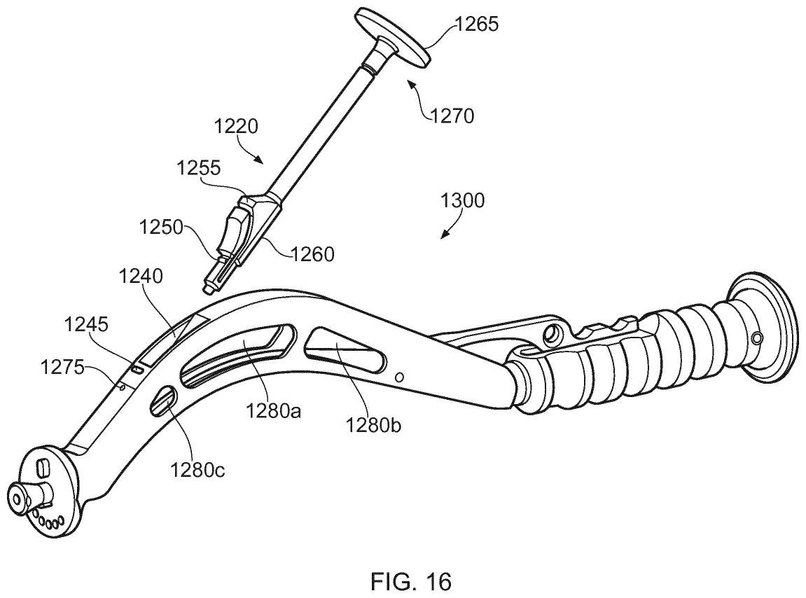

The handle 1220 may be secured to the base body by any suitable fixing means. In the embodiment shown in FIG. 16, the base body comprises a groove 1240 into which a distal end of the handle may be located. The base body comprises generally parallel walls which together define the groove and which facilitate the location of the handle into the groove.

The base body further comprises an aperture 1245 on an upper surface thereof. The aperture is sized to accommodate a protrusion 1250 located on the handle. The protrusion 1250 is spring loaded so it can be depressed as the handle is driven into the groove and then located and secured in the aperture. A user may depress the protrusion and pull the handle away from the base body in order to detach the handle from the base body.

The handle also comprises a thickened portion 1255 which provides a stop to limit movement of the handle along the groove of the base body. As shown in FIG. 16, the protrusion is spring loaded by way of the provision of a channel 1260 within the thickened portion.

The handle may comprise a flattened end portion 1265 at a proximal end region 1270. The handle 1220 is used by the surgeon to manoeuvre the introducer apparatus during surgery.

In certain embodiments, the base body further comprises a second aperture 1275 located distally from the aperture. The second aperture is provided to enable the groove and the elongate body portion generally to be washed and flushed out thus improving the cleanability of the base body.

The embodiments of FIGS. 11E-H and 16 include a base body which includes at least one cut out region 1280a, 1280b, 1280c. The provision of cut out regions reduces the weight of the introducer apparatus and therefore makes the apparatus easier to handle.

In certain embodiments, the introducer apparatus is also used as an impactor apparatus.

The embodiment shown in FIGS. 11G and H shares many of the features of the apparatus of FIG. 11E and F. However, the apparatus 1300 of FIG. 11G and FIG. 11H is for use in a cementless procedure. As such, the lever portion 1310 is a self-locking lever arm as described above.

In certain embodiments, there is provided a system comprising a device as described above. The device may also be referred to as an introducer plate. The system may further comprise a positioning apparatus as described herein. The positioning apparatus may also be referred to herein as an introducer.

### Method of Surgery

In certain embodiments, apparatus, devices and methods described herein are for use in surgery to implant a POLARCUP® acetabular component manufactured by Smith & Nephew.

In preparation for the method of hip surgery according to certain embodiments of the present invention, X-rays of the patient's pelvic region may be taken. In certain embodiments, preoperative X-Rays should include an anterior-posterior projection (AP) of the pelvis centered over the symphysis and an AP and lateral projection of the affected hip. Aptly, templating can be done on the affected side, but it is important that the contralateral hip also be templated to verify the size. To ensure a congruent fit, the acetabular component should be medialized to the medial aspect of the acetabulum, as indicated by the teardrop. The center of rotation also should be marked for subsequent reference.

In certain embodiments, the acetabulum of the patient is completely exposed. Approaches may vary between surgeons but in one embodiment, the method first comprises resecting the acetabular labrum. A blunt retractor may be placed anteriorly. After the transverse acetabular ligament is identified, a blunt retractor can be placed around the inferior margin of the acetabulum. Depending on the exposure, a third retractor can be placed posteriorly following the excision of the labrum. Overhanging soft tissue and osteophytes are removed in order to visualize the entire acetabular socket. The acetabulum should be medialized to restore the normal center of hip rotation.

Aptly the surgery is performed with the patient in an extended supine or lateral position. Access to the operative site is based on previously recorded patient data or the preference of the operating surgeon(s).

The joint capsule may be opened and, if necessary, completely resected. Aptly the acetabulum is then exposed to provide a good view and create sufficient space for the reaming instruments. The osteophytes from the edge of the hip joint cup and the acetabular fossa are then removed followed by cleaning of the hip joint cup.

The acetabulum is then prepared with a reamer, avoiding excessive reaming on the periphery to avoid bone loss. Reaming is continued with increasing diameters (in 2 mm steps), taking into consideration the final anteversion and inclination, until the cartilage is completely removed.

For cemented cups, the last reamer size used should be at least two sizes larger than the implanted size (e.g. size 55 mm reamer=size 51 mm implant) which produces a layer of cement of approximately 2 mm all around.

In certain embodiments, the apparatus as described herein is used to align cemented prostheses. Aptly, the device for introducing the prosthesis is placed at the distal end of the apparatus, with the protrusion of the apparatus being positioned through the hole of the device (also referred to herein as “introducer plate”) until the Introducer Plate touches the metal plate of the apparatus. The Introducer Plate is then pressed against the protrusion of the apparatus until a click is heard.

The wing portions of the Introducer Plate are snapped over the protrusion.

The acetabulum is washed out and dried before applying the cement. An acetabular prosthesis of the appropriate size is then placed on the Introducer plate which is in turn attached to the apparatus. In certain embodiments, the prosthesis comprises a notch or other visual indicator which can be aligned with a corresponding visual indicator e.g. a notch located on the Introducer Plate.

The prosthesis can then be placed into the wound, by holding it on the apparatus by hand. In certain embodiments, the combination of the apparatus and introducer plate (device) as described herein allows a loose connection between the introducer plate and the acetabular component but also fully covers the acetabular component to prevent cement inflow.

Furthermore, the introducer plate as described herein (also referred to as a device) enables fixation of an acetabular component for use with cement with a strong retention force when introducing and aligning the prosthesis but is completely loose from the prosthesis when the apparatus is released and withdrawn from the acetabular component when in situ in the surgical site.

The acetabular component is inserted into the acetabulum by placing the 6° cover in a superior posterior position and pressing the acetabular component into the cement. In certain embodiments, the acetabular component is then aligned into 45° inclination and 20° anteversion by using the connection between the Introducer Plate and the acetabular component rim.

20° anteversion: For right hips the first branch portion may be held in parallel to the coronal plane of the patient. Aptly, in certain embodiments the first branch portion comprises a visual indicator to indicate it is for use to align an acetabular component intended to be placed in the right hip of the patient. Aptly, the further branch portion may be provided with a visual indicator to indicate that it is for use to align an acetabular component in a left hip. Aptly, for left hips the branch portion labelled with “left” is used and held parallel to the coronal plane of the patient. For right hips the arm labelled with “right” is used.

45° inclination: in order to align the acetabular component at 45° inclination, the apparatus is held such that the arm portion is at a 90° position to the patients' axis when the patient is in a supine or lateral position on an operating table. Once the surgeon is content that the acetabular component is in the correct position with respect to inclination and anteversion, the cup is then fixed in place by the surgeon. The apparatus is withdrawn from the wound site.

The surgeon then completes the surgery and closes the wound.

In certain embodiments, the acetabular component is an acetabular shell or cup intended for use with a fixation material such as cement. In other embodiments, the acetabular component is a trial prosthesis e.g. a trial acetabular component. In yet further embodiments, the acetabular component may be an acetabular shell. The shell may comprise pegs and/or flanges for example. The acetabular component may be formed from known materials suitable for implants e.g. titanium, stainless steel, chromium, cobalt or alloys thereof.

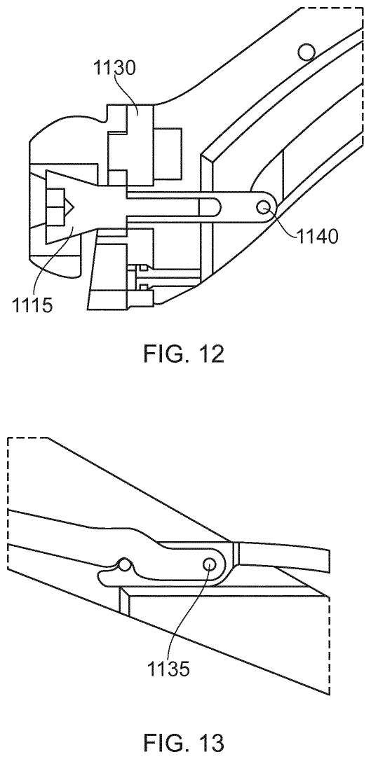

In certain embodiments, the introducer plate can be used to introduce non-cemented acetabular components in a prepared acetabulum of a patient. For example, a curved or straight introducer apparatus can be used to place the acetabular component into the surgical site. Suitable introducer apparatus are described herein and shown in FIGS. 11 to 15.

As noted above, in certain embodiments, the introducer plate can be used with an Introducer apparatus as described herein for cemented implantation of an acetabular component. For example, the introducer plate can be coupled with an apparatus which comprises an inner lever which is not a self-locking lever. In this embodiment, the inner-lever needs to be held by a user as they grip the handle. The inner lever will be released (open) when the user removes their hand from the handle. When the inner lever is released, this causes retraction of the wing portions of the introducer plate which is located at the distal end of the apparatus. The retraction of the wing portions enables an acetabular component located on the introducer plate to be released.

This embodiment is particularly useful when implanting with cement, as the acetabular component must be held still. When using a self-locking inner-lever, the release of the shell is only possible by unlocking the self-locking mechanism, an action which causes little shocks which are not acceptable for implantation with cement. Thus, a non-self-locking mechanism allows a very smooth and controlled release of the shell without little shocks.

In embodiments in which acetabular components are implanted without cement, the introducer apparatus may comprise a self-locking inner lever as described above. The self-locking inner lever enables an acetabular component which is mounted on the introducer plate which is in turn mounted on the central protrusion of the apparatus to be retained. The user can take their hand away from the handle and still retain the acetabular component on the introducer plate.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. An apparatus for locating an acetabular component in an acetabulum of a patient, the apparatus comprising
a. a proximal portion;
b. a distal portion; and
c. a connecting portion intermediate the proximal portion and the distal portion, wherein the connecting portion includes:
i) an arm portion connected to the distal portion and comprising a longitudinal axis; and
ii) an alignment guide portion extending between the proximal portion and the arm portion, the alignment guide portion being integrally formed with the proximal portion and the distal portion and comprising a first branch portion and a second branch portion, and wherein the first branch portion and/or the second branch portion provides an alignment surface.

2. The apparatus according to claim 1, wherein the acetabular component comprises a polar axis and the apparatus serves to orient the polar axis at a prescribed angle of inclination and a prescribed angle of anteversion.

3. The apparatus according to claim 1, wherein when the patient is located in the supine or lateral position on an operating table, the patient having a long axis, the alignment surface is configured to be beatable at a predetermined angle with respect to the long axis of the patient so as to orient the polar axis at the prescribed angle of inclination.

4. The apparatus according to claim 1, wherein the alignment surface comprises a plane and wherein the alignment surface is generally parallel to the coronal plane of the patient to represent an angle of anteversion of between 10° to 20°.

5. The apparatus according to claim 1, wherein the first branch portion comprises a first alignment surface configured to align the acetabular component in a first acetabulum of the patient and the second branch portion comprises a further alignment surface configured to align the acetabular component in a further acetabulum of the patient.

6. The apparatus according to claim 1, wherein the apparatus serves to orient the acetabular component along an alignment axis in the acetabulum during surgery, wherein the alignment axis is an axis along which it is desirable to insert an acetabular component into the acetabulum during surgery.

7. The apparatus according to claim 6, wherein the alignment axis extends at an angle of:
about 40 to 50° inclination from the centre of the acetabulum; and
an angle of 0 to 30° anteversion from the centre of the acetabulum.

8. The apparatus according to claim 1, wherein the alignment guide portion comprises a central region from which the first branch portion and the second branch portion extend at an acute angle to each other, the acute angle being in the range of from about 15° to about 60°.

9. The apparatus according to claim 8, wherein the acute angle is about 35° to about 45°.

10. The apparatus according to claim 8, wherein the acute angle is about 40°.

11. The apparatus according to claim 1, wherein the first branch portion and the second branch portion are substantially symmetrical about a longitudinal axis of the alignment guide portion.

12. The apparatus according to claim 1, wherein the arm portion comprises a longitudinal axis and further wherein the longitudinal axis of the alignment guide portion and the longitudinal axis of the arm portion are generally orthogonal with respect to each other.

13. The apparatus according to claim 1, wherein the proximal portion comprises a handle portion at a proximal end thereof.

14. The apparatus according to claim 1, wherein the distal portion comprises an insertion portion at a distal end thereof, wherein the insertion portion comprises a plate portion having a central protrusion.

15. The apparatus according to claim 14, wherein the plate portion is generally circular.

16. The apparatus according to claim 14, wherein the central protrusion comprises a conical frustum.

17. The apparatus according to claim 1, wherein the apparatus is an integrally formed apparatus.

18. The apparatus according to claim 1, wherein the apparatus further comprises a detachable introducer plate configured to be beatable in an acetabular component.

19. The apparatus according to claim 1, wherein the apparatus is configured to locate a cemented acetabular component in the acetabulum of a patient.

20. Apparatus for introducing an acetabular component into an acetabulum of a patient, the apparatus comprising:
i) a proximal portion comprising a gripping portion;
ii) a distal portion comprising a securing element comprising a frusto-conical surface; and
iii) an elongate body portion extending between the proximal portion and the distal portion, the elongate body portion comprising a lever element pivotally secured to the elongate body portion, wherein the lever element is pivotable to drive the frusto-conical surface of the securing element between a first position away from the elongate body portion for receiving an acetabular component and/or an introducing plate as described herein and a further position in which the frusto-conical surface is located proximate to an end of the elongate body portion to secure an acetabular component and/or an introducing plate as described herein at an end of the elongate body portion.

21. The apparatus according to claim 20, wherein the elongate body portion further comprises a drive arm pivotably connected to the lever element and to the securing element.

22. The apparatus according to claim 20, wherein the drive arm is housed within the elongate body portion.

23. The apparatus according to claim 20, wherein the elongate body portion comprises a cylindrical extension at a distal end thereof.

24. The apparatus according to claim 20, wherein the elongate body portion further comprises at least one cut-out portion.

25. The apparatus according to claim 20, further comprising a handle element connectable to the elongate body portion, wherein the handle portion comprises a hexagonal end portion.

26. The apparatus according to claim 20, wherein the elongate body portion is arcuate or is substantially straight.

27. The apparatus according to claim 20, wherein the lever element comprises an arcuate portion and a flattened portion, wherein the flattened portion is configured to locate at least partially within a recessed portion of the grippable portion or the lever element comprises a straight portion and a flattened portion wherein the flattened portion is configured to locate at least partially within a recessed portion of the grippable portion.

28. A method of locating an acetabular component in an acetabulum of a patient, the method comprising:
a). providing an apparatus according to claim 20;
b) locating an acetabular component at the distal end of the apparatus; and c) advancing the apparatus to locate the acetabular component in the acetabulum of the patient.

29. The method according to claim 28, further comprising locating a fixation material in the acetabulum of the patient prior to step (c).

30. The method according to claim 28, further comprising providing the apparatus according to claim 1, aligning the first branch region or the second branch region such that the plane of the respective alignment surface is generally parallel to the coronal plane of the patient, when the patient is positioned in an extended supine or lateral position.

31. The method according to claim 28, further comprising aligning the arm portion generally orthogonal to the longitudinal axis of the patient, when the patient is positioned in an extended supine or lateral position.

32. The method according to claim 28, further comprising withdrawing the apparatus from the acetabulum once the acetabular component has been located in a desired orientation in the acetabulum.

33. The method according to claim 28, further comprising, prior to step (c), the steps of:
a) incising a hip with an incision; and
b) exposing at least a portion of an acetabular joint through the incision.

34. A kit comprising the apparatus according to claim 1 or the apparatus according to claim 20 and at least one acetabular component.

35. A kit according to claim 34, wherein the acetabular component is a trial component or an acetabular prothesis.

36. A kit according to claim 34, further comprising a fixation material.

37. A device for introducing an acetabular component into an acetabulum of a patient; the device comprising:
a) a base portion comprising a central aperture;
b) a radially expandable upper portion having a lower surface which is at least partially connected to the base portion, the upper portion comprising at least one slit and a central cut-out region, wherein the cut out region of the upper portion and the central aperture of the base portion are at least partially aligned.

38. A device according to claim 37, wherein the at least one slit is configured to enable the upper portion to expand or contract in circumference upon application of pressure.

39. A device according to claim 37, wherein the upper portion comprises at least two slits.

40. A device according to claim 37, wherein the upper portion is a truncated cylinder.

41. A device according to claim 37, wherein the base portion comprises an outer annular rim portion.

42. A system comprising the apparatus according to claim 1 or the apparatus according to claim 20 and a device according to claim 37, wherein the central protrusion is configured to be beatable in the central aperture of the base portion and the cut out region of the upper portion.

43. A system according to claim 42, wherein the device is an introducer plate.
